(19) United States
(12) Patent Application Publication
(10) Pub. No.: US 2018/0002535 A1
(43) Pub. Date: Jan. 4, 2018

(54) A METHOD FOR TREATING A SURFACE AND AN ARTICLE COMPRISING A LAYER OF MICROBIAL STRUCTURES

(71) Applicant: KONINKLIJKE PHILIPS N.V., EINDHOVEN (NL)

(72) Inventor: SHUANG CHEN, EINDHOVEN (NL)

(21) Appl. No.: 15/546,835

(22) PCT Filed: Jan. 11, 2016
(86) PCT No.: PCT/EP2016/050325

§ 371 Date: Jul. 27, 2017

(30) Foreign Application Priority Data
Jan. 28, 2015 (CN) PCT/CN2015/071680
Mar. 10, 2015 (EP) 15158521.3

(51) Int. Cl. C09D 5/16; C12N 1/20

(52) U.S. Cl. CPC C09D 5/1681; C12N 1/20

(57) ABSTRACT

The present invention provides a method of treating a surface of a substrate, the method comprising: a) growing a microbe on the surface of the substrate to be treated to form a layer of microbial structures, wherein the microbe is selected from fungi, algae, lichens and any combination thereof; and b) coating the microbial structures to form a first coating thereon, wherein the first coating has a thickness of no more than 1 μm. The present invention also provides an article comprising a layer of microbial structures, optionally made by using the method of the present invention.

### FIELD OF THE INVENTION

The present invention relates to a method of treating a surface of a substrate.

The present invention also relates to an article comprising a layer of microbial structures.

### BACKGROUND OF THE INVENTION

Surface treatment is very common in providing a desired surface property or a desired surface topography. Like disclosed in “Self Assembling hydrophobic biofilm”; —& “Biofilm”; —& “Chaplins”, Sep. 4, 2014, a kind of bacterium is used to form a biofilm on a substrate first, and then the biofilm producing hydrophobic protein to form a hydrophobic surface. A kill switch to kill the bacterium afterward but the hydrophobic protein remains. By this method, the treated surface shows a hydrophobic character.

Another approaches for surface treatment is biomimetics. It is well established that many surfaces in nature can lead to specific behavioral phenomena. Many methods have been developed to mimick the morphology of a natural surface so as to achieve the desired surface property or topography.

Water/oil repellency property is one of the important properties which can be provided by mimicking the morphology of a natural surface and therefore draw a lot of attention in daily life as well as in many industrial and biological processes. Examples of such natural surfaces include those found on the leaves of some plants, such as the lotus, the legs of the water striders, and the wings of some insects exhibiting the unusual phenomenon of superhydrophobicity. One famous example is the leave of the lotus, which was found to be superhydrophobic a long time ago. The feature of these surfaces is that they usually have binary structures on the micrometer and nanometer scales, which results in large water contact angles. This is because air can be trapped between the water droplets and the wax crystals at the surface, which minimizes the real contact area of the water droplets with the surfaces. The application of the water/oil repellency property includes, for example, reduction of frictional drag on ship hulls, anti-icing and self-cleaning surfaces. As regards transparent hydrophobic surfaces, the range of possible applications could be expanded to glass-based substrates such as goggles or windshields.

Artificial superhydrophobic surfaces with water contact angles larger than 150° have been prepared by replicating the surface topography of natural superhydrophobic surfaces with the aid of various processing techniques such as etching and machining. Many artificial superhydrophobic surfaces have also been investigated with regard to their wetting dynamics as well as their chemical, mechanical and thermal stability, opening many applications in industrial and biological processes. However, many of the current processing techniques for fabricating artificial superhydrophobic surfaces are difficult and typically use expensive materials or severe conditions. Together with poor stability and short lifetime, these issues largely limit the application of artificial superhydrophobic surfaces.

Therefore, there is a need to develop a simple, low-cost method for surface treatment by mimicking the morphology of a natural surface. There is also a need to provide a robust superhydrophobic/superoleophobic surface in a convenient way.

### SUMMARY OF THE INVENTION

In a first aspect, the present invention relates to a method of treating a surface of a substrate, the method comprising:

a) growing a microbe on the surface of the substrate to be treated to form a layer of microbial structures, wherein the microbe is selected from fungi, bacteria, algae, lichens and any combination thereof; and
b) coating the microbial structures to form a first coating thereon, wherein the first coating has a thickness of no more than 1 μm.

In a second aspect, the present invention relates to an article, comprising a substrate, a layer of microbial structures formed on at least a portion of a surface of the substrate, and a first coating formed on the microbial structures, wherein the microbial structures comprise a structure originating from fungi, bacteria, algae, lichens or any combination thereof; and wherein the first coating has a thickness of no more than 1 μm.

### BRIEF DESCRIPTION OF THE DRAWINGS

For the purpose of illustrating the invention, the following drawings are provided without any intention to limit the invention.

FIG. 1 is a schematic depiction of an article comprising the surface being treated by the method of the present invention.

FIG. 2a and FIG. 2b, in different magnifications, illustrate the water droplet on the surface of streptomyces albus cultured on the surface of a glass culture dish according to an embodiment of the present invention.

FIG. 3a, FIG. 3b and FIG. 3c, in different magnifications, illustrate the water droplet on the surface of a superhydrophobic coating obtained according to an embodiment of the present invention.

FIGS. 4a-4d illustrate a peeling test, wherein FIG. 4a shows the adhesive tape used for peeling the superhydrophobic coating and the peeling area of the superhydrophobic coating (marked in a rectangle) in the peeling test, and FIGS. 4b, 4c and 4d show the status of the water droplet on the surface of the superhydrophobic coating after 1, 5 and 10 peeling operations, respectively.

FIG. 5a shows the sample preparation of the superhydrophobic coating obtained according to an embodiment of the present invention for scanning electron microscope (SEM) analysis. FIGS. 5b and 5c show the SEM images of the surface of the superhydrophobic coating obtained, in different magnifications.

### DETAILED DESCRIPTION OF THE EMBODIMENTS

Although the present invention will be described with respect to particular embodiments, this description is not to be construed in a limiting sense.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5% or even ±1%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is an object of the present invention to provide a simple and low-cost method for surface treatment by mimicking the morphology of a natural surface. One of the advantages of the present invention lies in that the artificial surface obtained by the method of the present invention is robust and stable.

According to the present invention, microbes are used for surface treatment. In spite of the fact that growth of microbes on a surface is generally undesirable and tends to be prevented or avoided, it has surprisingly been found that growth of microbes on a surface can help to impart desired properties to the surface in a robust way.

In an embodiment, the present invention provides a method of treating a surface of a substrate, the method comprising:

a) growing a microbe on the surface of the substrate to be treated to form a layer of microbial structures, wherein the microbe is selected from fungi, bacteria, algae, lichens and any combination thereof; and
b) coating the microbial structures to form a first coating thereon, wherein the first coating has a thickness of no more than 1 μm.

In another embodiment, the present invention provides an article, comprising a substrate, a layer of microbial structures formed on at least a portion of a surface of the substrate, and a first coating formed on the microbial structures, wherein the microbial structures comprise a structure originating from fungi, bacteria, algae, lichens or any combination thereof; and wherein the first coating has a thickness of no more than 1 μm.

In a further embodiment, the present invention provides an article made by using the method of the present invention for treating at least a portion of a surface of a substrate of the article.

### Microbe and Microbial Structure

The term “microbe” or its plural form “microbes”, as used herein, refers to any small or tiny living thing, including a plant and an animal in a broad sense. It is intended as a collective term for the large variety of microorganisms, including all of the prokaryotes, namely the bacteria and archaea; and various forms of eukaryotes, comprising the protozoa, fungi, algae, microscopic plants (green algae); and animals such as rotifers and planarians; and mutualistic combinations of single microbes as listed above, such as lichens.

The term “fungus” or its plural form “fungi”, as used herein, refers to any member of a large group of eukaryotic organisms that includes microorganisms such as yeasts and molds, mildews, rusts, and yeasts.

The term “alga” or its plural form “algae”, as used herein, refers to any member of a very large and diverse group of simple, typically autotrophic organisms, ranging from unicellular to multicellular forms.

The term “lichen” or its plural form “lichens”, as used herein, refers to any of certain plants formed by the mutualistic combination of an alga and a fungus.

The term “microbial structure” or its plural form “microbial structures”, as used herein, refers to a structure originating from one or more microbes. In other words, a microbial structure is a structure formed from the growth of one or more microbes.

The term “a layer of microbial structures”, as used herein, refers to a layer which comprises microbial structures or a profile of microbial structures. In other words, a layer of microbial structures may be a layer in which the one or more microbes forming the microbial structures remain after the layer has been formed, or it may be a cavity which has the same profile as the microbial structures but from which the one or more microbes forming the microbial structures have been removed.

According to the present invention, the microbe which can be suitably used includes species selected from fungi, bacteria, algae, lichens and any combination thereof. These microbes can grow on a surface under specific conditions to form a layer of microbial structures. This is advantageous since it provides the possibility of forming a robust connection between the layer of microbial structures and the surface. On the other hand, the microbial structures generally have delicate and complex micro- and nanoscale surface architectures which are desired for certain surface properties, such as superhydrophobicity and/or superoleophobicity. The use of a microbe as a template for producing a superhydrophobic coating is very novel and has not been reported previously. In the case of using a microbe as the template, further etching or another kind of surface architecture building would be optional or even can be waived.

In a specific embodiment, the microbe is selected from fungi or bacteria comprising hyphae with a diameter of between 0.3 μm and 100 μm. A hypha is a long, branching filamentous structure of a fungus. In most fungi or bacteria, hyphae are the main mode of vegetative growth. In a preferred embodiment, the microbe is selected from fungi or bacteria comprising hyphae with a diameter of between 0.3 μm and 70 μm, preferably between 0.5 μm and 50 μm, more preferably between 1 μm and 25 μm, most preferably between 1.5 μm and 15 μm. Suitably, the hyphae can have a diameter in the range of between 55 μm and 100 μm, or between 60 μm and 90 μm, or between 0.3 μm and 40 μm, or between 0.8 μm and 10 μm. The presence of hyphae makes it possible to form intertwined and entangled structures which enable microscopic pockets of air be trapped therein and thereby support water/oil drops to stand thereon or to roll off.

Suitably, the fungi or bacteria may be selected from Molds, Actinomycetes and any combination thereof. Examples of the fungi or bacteria which can be suitably used in the present invention include, but are not limited to, Rhizopus, Mucor, Neurospora, Aspergillus, Penicillium, Streptomyces, Nocardia, Frankia, Actinoplanes, thermomonospora and any combination thereof. In a specific embodiment, Streptomyces is used as the microbe in the method of the present invention. The Streptomyces may be suitably selected from Streptomyces albus, Streptomyces griseus, Streptomyces venezuelae, Streptomyces aureofaciens and any combination thereof.

Suitably, the algae may be selected from Archaeplastida including Chlorophyta (green algae), Rhodophyta (red algae), Glaucophyta, Charophyta; Rhizaria (Excavata) including Chlorarachniophytes, Euglenids; Chromista (Alveolata) including Heterokonts (such as Bacillariophyceae (Diatoms), Axodines, Bolidomonas, Eustigmatophyceae, Phaeophyceae (brown algae), Chrysophyceae (golden algae), Raphidophyceae, Synurophyceae, and Xanthophyceae (yellow-green algae)), Cryptophyta, Dinoflagellata, Haptophyta; Cyanobacteria (blue-green algae); and any combination thereof.

Suitably, the lichens may be selected from fruticose, foliose, crustose, leprose, gelatinous, filamentous, Byssoid, structureless, and any combination thereof.

Optionally, a growth medium or culture medium may be suitably used to help the microbe grow on the surface. Generally, a growth medium or culture medium is a liquid or gel designed to support the growth of microbes. The most common growth media for microbes are nutrient broths and agar plates. Those skilled in the art would be able to select a suitable medium for the specific microbes to be used.

The layer of microbial structures may have a thickness of any value which can suitably enable the achievement of the advantageous technical effect of the present invention. In a specific embodiment, the layer of microbial structures may have a thickness of from 1 μm to 10 mm, preferably from 10 μm to 5 mm, more preferably from 100 μm to 3 mm, most preferably from 200 μm to 1 mm, 300 μm to 1.5 mm, or from 400 μm to 2 mm, or from 120 μm to 600 μm.

The thickness of the layer of microbial structures can be tuned by the culture time. The culture time may be from several minutes to several months, depending on the specific microbe to be used. Generally, a microbe which can form a layer of microbial structures of the above thickness during the culture time of several minutes or up to 1-4 days would be desired. Those skilled in the art would be able to select the suitable culture time and environment conditions for the specific microbes to be used.

Optionally, a step of drying the microbial structures can be conducted after the desired thickness is achieved. The drying process may last 0.5-24 hours, for example 0.5-2 hours, 5-10 hours or 12-24 hours.

Suitably, the method of the present invention may further comprise a step of deactivating or removing the microbe after forming the first coating. The step of deactivating or removing the microbe, also called a sterilization step, may be achieved by applying heat, chemicals, irradiation, high pressure, filtration, or any combination thereof. Heat sterilization includes dry heat sterilization and moist heat sterilization, for example, steam sterilization, autoclave treatment, flaming, incineration, boiling or the like. Examples of chemicals which can be used for sterilization include, but are not limited to, ethylene oxide, nitrogen dioxide, ozone, bleach, glutaraldehyde and formaldehyde solutions, phthalaldehyde, hydrogen peroxide, peracetic acid, silver, and ZnO. Radiation sterilization may use radiation such as electron beams, X-rays, gamma rays, or subatomic particles. Those skilled in the art would be able to select a suitable sterilization method for deactivating or removing the microbe within the spirit of the present invention.

### The First Coating

By coating the microbial structures to form a first coating thereon, it is possible to replicate and fix the desired surface architectures.

For the purpose of the present invention, the first coating typically has a thickness of no more than 1 μm. The thickness of the first coating may vary with the specific microbe used and the microbial structures formed thereby. In a specific embodiment, the first coating has a thickness of no more than 0.9 μm, suitably no more than 0.5 μm, more suitably no more than 0.1 μm, most suitably no more than 0.05 μm. In a specific embodiment, the first coating has a thickness of no less than 1 nm, suitably no less than 5 nm, more suitably no less than 10 nm, most suitably no less than 15 nm. The first coating may have a thickness in the range from 8 nm to 80 nm, or from 12 nm to 20 nm, or from 30 nm to 45 nm, or from 60 nm to 120 nm. The thickness of the first coating should enable the first coating to cover the microbial structures on the one hand and replicate the surface architectures of the microbial structures on the other hand. The thickness of the first coating could be suitably selected to maximize the advantageous technical effect of the present invention.

The first coating can be formed on the microbial structures by any suitable method. For example, one or more methods selected from vapor deposition, self-propagation high-temperature synthesis, thermochemical synthesis, thermal spray, electrochemical synthesis, sol-gel process, and in situ formation, casting may be used to form the first coating.

The material of the first coating may be organic or inorganic, provided that it can form a shell around the microbial structures. The term “shell”, as used herein, refers to an external layer covering at least part of the microbial structures. By covering at least part of the microbial structures, the shell has an inner surface topography complementary to the surface of the layer of the microbial structures, and an outer surface replicating the micro- and nano-scale architectures on the surface of the layer of the microbial structures. The shell is preferably hard enough to be self-supported and/or resistant to scratching, so that the surface topography or the micro- and nanoscale architectures obtained can be retained in a durable way. In a specific embodiment, the shell covers all the microbial structures in such a way that a cavity which has the same profile as the microbial structures will be formed between the substrate and the shell once the one or more microbes forming the microbial structures are removed.

In a specific embodiment, the first coating is an inorganic coating, for example, consisting of an inorganic oxide selected from silicon oxides, magnesium oxides, aluminum oxides, zinc oxides, iron oxides, copper oxides, silver oxides, titanium oxides, and any combination thereof.

In another specific embodiment, the first coating is an organic coating, for example consisting of a film-forming polymer or composition. Examples of the film-forming polymer include, but are not limited to, cellulose-based polymers such as nitrocellulose, cellulose acetate, cellulose acetobutyrate, cellulose acetopropionate and ethylcellulose; polyurethanes; acrylic polymers; vinyl polymers; polyvinyl butyrals; alkyd resins; resins derived from aldehyde condensation products such as arylsulfonamide-formaldehyde resins, for instance toluenesulfonamide-formaldehyde resin, arylsulfonamide-epoxy resins or ethyltosylamide resins. The film-forming composition may advantageously comprise at least one film-forming polymer, and optionally at least one auxiliary film-forming agent.

### The Second Coating

The method of the present invention may further comprise a step of forming a second coating on the first coating. The second coating may work with the microbial structures, especially the surface architectures of the microbial structures, to provide a synergetic effect. For example, the second coating may comprise one or more functionalized materials, such as hydrophobic materials, oleophobic materials, or amphiphobic materials to provide a superhydrophobic surface, a superoleophobic surface, or a superamphiphobic surface. Any materials, including organic materials and inorganic materials, with desired properties can be used for the purpose of the present invention.

In a specific embodiment, the second coating has a surface energy of no more than 70 mJ/m². In a preferred embodiment, the second coating has a surface energy of no more than 60 mJ/m², preferably no more than 50 mJ/m², more preferably no more than 40 mJ/m², most preferably no more than 30 mJ/m². For the purpose of the present invention, the second coating can have a surface energy which is as low as possible. For example, the surface energy of the second coating may be as low as 10 mJ/m², 15 mJ/m², or 20 mJ/m².

Examples of the materials used for forming the second coating include, but are not limited to, polyhexafluoropropylene (PHFP), polytetrafluoroethylene (PTFE/Teflon), fluorinated ethylene propylene (FEP), polytrifluoroethylene, chlorotrifluoroethylene (Aclar), polydimethyl siloxane (silicone elastomer), fluorinated silane such as trichloro (1H,1H,2H,2H-perfluorooctyl)silane, natural rubber, paraffin, polyvinylidene fluoride (PVDF), polyvinyl fluoride (PVF/Tedlar), polypropylene (PP), polyethylene (PE), polychlorotrifluoroethylene (PCTFE), polybutylene terephthalate (PBT), Nylon-11 (polyundecanamide), surlyn ionomer, polystyrene (PS), polyacrylate, polyvinyl chloride (PVC), polyvinyl alcohol (PVOH/PVAL), polyphenylene sulfide (PPS), polyvinyl chloride (PVC), cellulose acetate (CA), polyvinylidene chloride (PVDC/Saran), polyimide (PI), polysulfone (PSU), polymethylmethacrylate (PMMA), Nylon-6 (polycaprolactam), polyethylene terephthalate (PET), regenerated cellulose, Nylon 6/6 (polyhexamethylene adipamide), polycarbonate (PC), polyphenylene oxide (PPO), styrene butadiene rubber, polyethersulfone, ethylene-vinyl acetate copolymer (EVA), polyurethane (PU), glass, silica, soda lime, copper, aluminum, iron, tin-plated steel, and any combination thereof.

In a specific embodiment, the second coating may be selected from an organofluorine coating, an organosilicone coating, a fluorosilicone coating, and any combination thereof.

For the purpose of the present invention, the second coating may have a thickness of no more than 2 μm. In a specific embodiment, the second coating has a thickness of no more than 1.5 μm, suitably no more than 1.0 μm, more suitably no more than 0.5 μm, most suitably no more than 0.1 μm. In a specific embodiment, the second coating has a thickness of no less than 5 nm, suitably no less than 10 nm, more suitably no less than 15 nm, most suitably no less than 20 nm. The second coating may have a thickness in the range from 8 nm to 80 nm, or from 60 nm to 120 nm, or from 100 nm to 200 nm, or from 300 nm to 800 nm. The thickness of the second coating could be suitably selected by those skilled in the art for the purpose of maximizing the advantageous technical effect of the present invention.

The second coating can be formed by any suitable method. For example, one or more methods selected from vapor deposition, self-propagation high-temperature synthesis, thermochemical synthesis, thermal spray, electrochemical synthesis, sol-gel process, and in situ formation, casting may be used to form the second coating.

### Substrate

The surface and/or the substrate which can suitably be treated by the method of the present invention may be made of any materials. In an embodiment, the surface and/or the substrate comprise one or more materials selected from glass, metal, ceramic, plastic, rubber, and textile. In a specific embodiment, the substrate to be treated is a textile. In another specific embodiment, the substrate to be treated is a glass or ceramic, optionally with a textile integrally formed on or adhered to the surface of said glass or ceramic. In a further specific embodiment, the substrate to be treated is a plastic or rubber, optionally with a textile integrally formed on or adhered to the surface of said glass or ceramic. Preferably, the substrate has a certain surface coarseness and/or porosity which facilitate the foundation of an environment for the one or more microbes growing on the surface of the substrate. Those skilled in the art would be able to select suitable surface coarseness and/or porosities based on the specific microbe to be used.

In an embodiment, the surface and/or the substrate to be treated exhibit a water contact angle smaller than 100°, or smaller than 90°, or smaller than 80°, or even smaller than 70°.

In an embodiment, the surface and/or the substrate, after treatment with the method of the present invention, exhibit a water contact angle greater than 150°, or greater than 155°, or greater than 160°, or even greater than 165°.

In a specific embodiment, the microbe forming the layer of microbial structures on the surface of the substrate comprises Streptomyces albus, the first coating comprises silicon dioxide, and the second coating comprises a fluorinated silane.

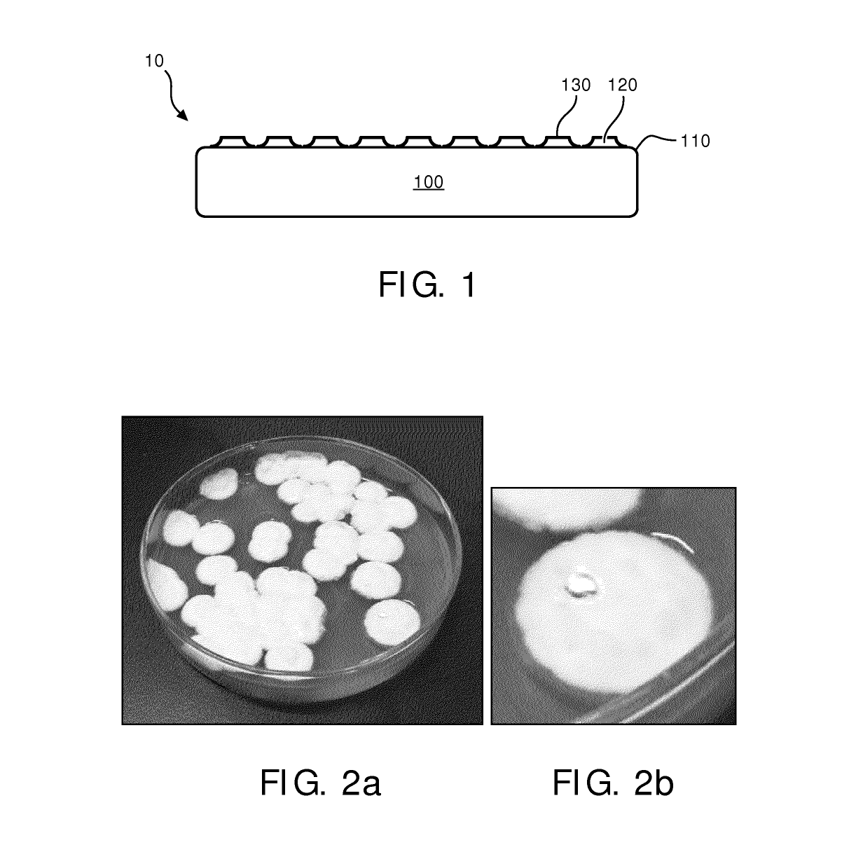

For the purpose of illustrating the present invention, FIG. 1 shows a schematic picture of an article 10 formed by the method of the present invention. The article 10 comprises a substrate 100, a layer of microbial structures 120 formed on at least a portion of a surface 110 of the substrate 100, and a first coating 130 formed on the microbial structures 120. A second coating (not shown) may be further formed on the first coating 130.

### Applications

The present invention is especially advantageous in providing a desired surface topography by mimicking a natural surface. By selecting a natural surface which has water/oil repellency properties, it is possible to fabricate a superhydrophobic/superoleophobic coating on the surface to be treated. Superhydrophobic/superoleophobic coatings could be applied to many industrial and biological processes, for example, reduction of frictional drag, anti-icing and self-cleaning processes. Further investigations into the application of superhydrophobic coatings in other areas such as air and kitchen have been proposed in the art.

Specifically, the present invention can find applications at least in the following aspects: superhydrophobic/superoleophobic coating manufacturing; self-cleaning materials/surfaces; water-proof but gas permeable materials; air purifiers; air filtration; food/cosmetics storage; lighting applications such as a street lamp cooling system; healthcare applications such as a respironics mask or an artificial lung; and so on.

### EXAMPLES

The present invention will be further clarified by the following examples, which are intended to be purely exemplary of the invention. Other embodiments of the invention will be apparent to those skilled in the art from a consideration of this specification or practice of the invention as disclosed herein.

### I. Surface Treatment

A superhydrophobic coating was formed on the glass substrate with the use of Streptomyces albus to form the microbial structures. Streptomyces albus has complex micro- and nanoscale surface architectures showing a hydrophobic character.

Step 1: Formation of Microbial Structures Using agar as the culture medium, streptomyces albus was cultured on the surface of a glass culture dish for 3 days at room temperature (25° C.) to form a layer of microbial structures. The layer of microbial structures has a thickness of about 0.5 mm.

Put the glass culture dish along with the cultured streptomyces albus into a vacuum desiccator to dry the surface of streptomyces albus at about 70° C. for about 30 mins.

Step 2: Replication of the Surface Topography of the Microbial Structures

Chemical vapor deposition (CVD) of tetraethyl orthosilicate (TEOS) catalyzed by ammonia was carried out in the vacuum desiccator to deposit a SiO₂ layer on the surface of streptomyces albus. In this step, TEOS was hydrolyzed in the presence of the ammonium solution. After the orthosilicate was hydrolyzed, it formed SiO₂ on the surface of streptomyces albus. The function of the formed SiO₂ was to make a shell on top of streptomyces albus and allow for further superhydrophobic surface modification. The function of ammonium solution is to accelerate the hydrolysis of orthosilicate. The reaction during this step is shown in Scheme I below.

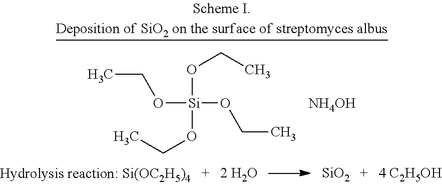

The thickness of the SiO₂ layer can be tuned by the duration of CVD. In this example, the CVD process lasts 24 hours and a SiO₂ layer of about 20 nm is formed around the streptomyces albus.

Step 3: Lowering the Surface Energy

Trichloro (1H,1H,2H,2H-perfluorooctyl)silane is used for lowering the surface energy of the SiO₂ layer by another CVD process. The thickness of the trichloro (1H,1H,2H,2H-perfluorooctyl)silane layer obtained in this step is about 20 nm. Scheme II shows the formula of Trichloro (1H,1H,2H,2H-perfluorooctyl)silane.

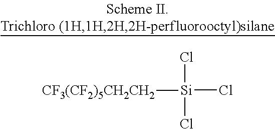

By means of the above three steps, a superhydrophobic coating was obtained on the surface of the glass culture dish.

### II. Measurement

The following experiments were conducted in order to demonstrate the technical effect of the above surface treatment.

### Water Contact Angle

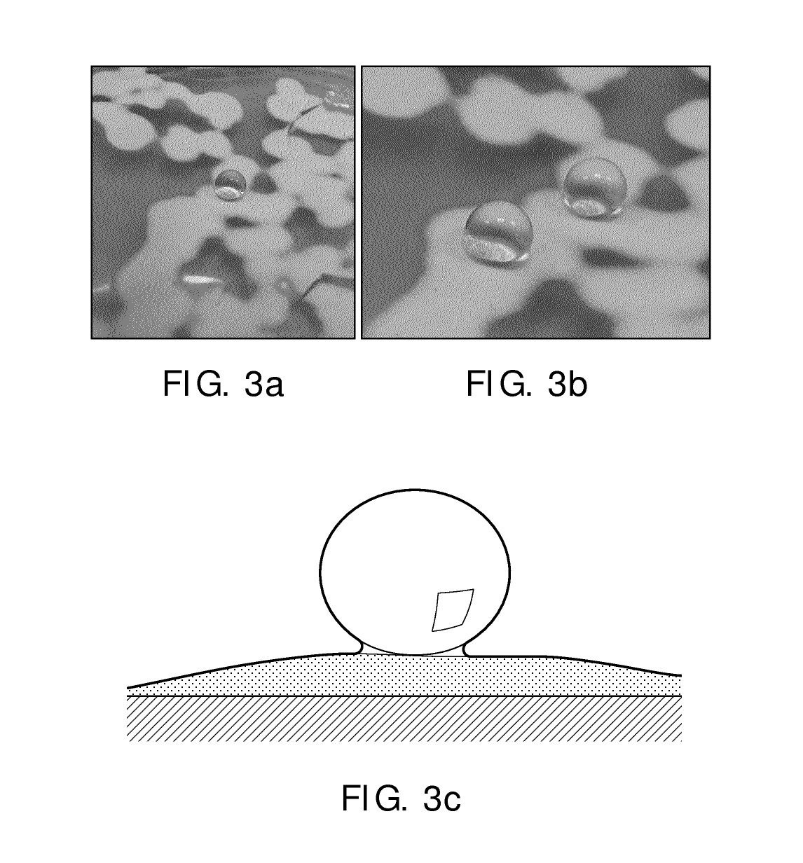

FIG. 2a and FIG. 2b, in different magnifications, illustrate the water droplet on the surface of streptomyces albus cultured on the surface of the glass culture dish. It can be seen that the water droplet stays on the surface of the streptomyces albus in the form of a small ball of water. However, the water contact angle is smaller than 90°.

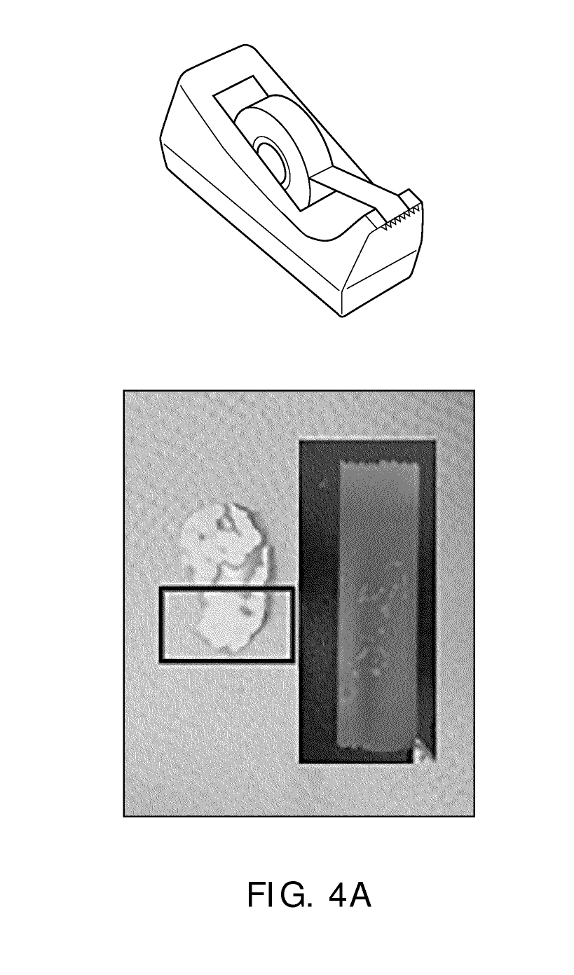

FIG. 3a, FIG. 3b and FIG. 3c, in different magnifications, illustrate the water droplet on the surface of the superhydrophobic coating as obtained above. It can be seen that the water droplets stand on the surface of the superhydrophobic coating in the form of small balls of water and exhibit a water contact angle larger than 150°. The contact angle as derived from FIG. 8c is about 150.41°.

Furthermore, from the comparison between FIGS. 2a-2b and FIGS. 3a-3c, it can be seen that a synergetic effect of the microbial structures and the conventional surface modification method is provided. In other words, even in the case that the microbial structures originating from streptomyces albus do not exhibit a superhydrophobic property, the use of the microbial structures has helped to fabricate a superhydrophobic coating by using a hydrophobic agent, trichloro (1H,1H,2H,2H-perfluorooctyl)silane.

Therefore, the present invention provides a new method of preparing a superhydrophobic coating by roughening the surface to be treated with the aid of the microbial structures and then lowering the surface energy of the roughened surface. In order to produce a superhydrophobic coating, a suitable surface architecture is essential. Previously, the methods often used wereetching and machining. The present invention makes it possible to eliminate the steps of etching and machining, and therefore provides a much easier and low-cost method for producing a superhydrophobic coating.

### Peeling Test

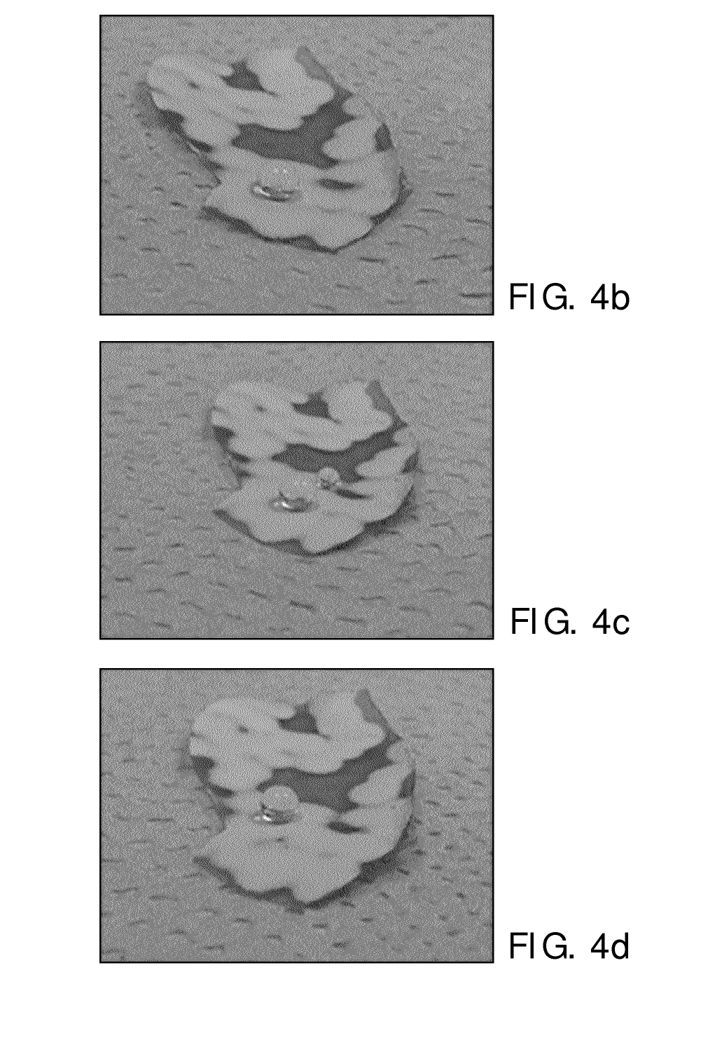

A peeling test was conducted by using an adhesive tape to demonstrate the robustness of the superhydrophobic coating as obtained above. FIG. 4a shows the adhesive tape used for peeling the superhydrophobic coating and the peeling area of the superhydrophobic coating (marked in a rectangle). The peeling test was conducted by the following peeling operation: at room temperature (25° C.), the adhesive side of the adhesive tape is brought into flat contact with the surface of the superhydrophobic coating with a pressure of 5 kg/cm² for 3 seconds, and then the adhesive tape is peeled off from the surface of the superhydrophobic coating. The same peeling operation was repeated 10 times. A water droplet was dropped on the superhydrophobic coating to observe the status of the water droplet after each peeling operation, and the superhydrophobic coating was dried in a vacuum desiccator prior to another peeling operation. FIGS. 4b, 4c and 4d show the status of the water droplet on the surface of the superhydrophobic coating after 1, 5 and 10 peeling operations, respectively.

It can be seen that, even after 10 peeling operations, the water droplet can still stand on the surface of the superhydrophobic coating in the form of a ball of water and exhibit a water contact angle larger than 150°. The superhydrophobic coating obtained by the present method is stable and robust, which presents opportunities for broad application of superhydrophobic coatings in many areas such as self-clean surfaces or devices.

### Scanning Electron Microscope (SEM)

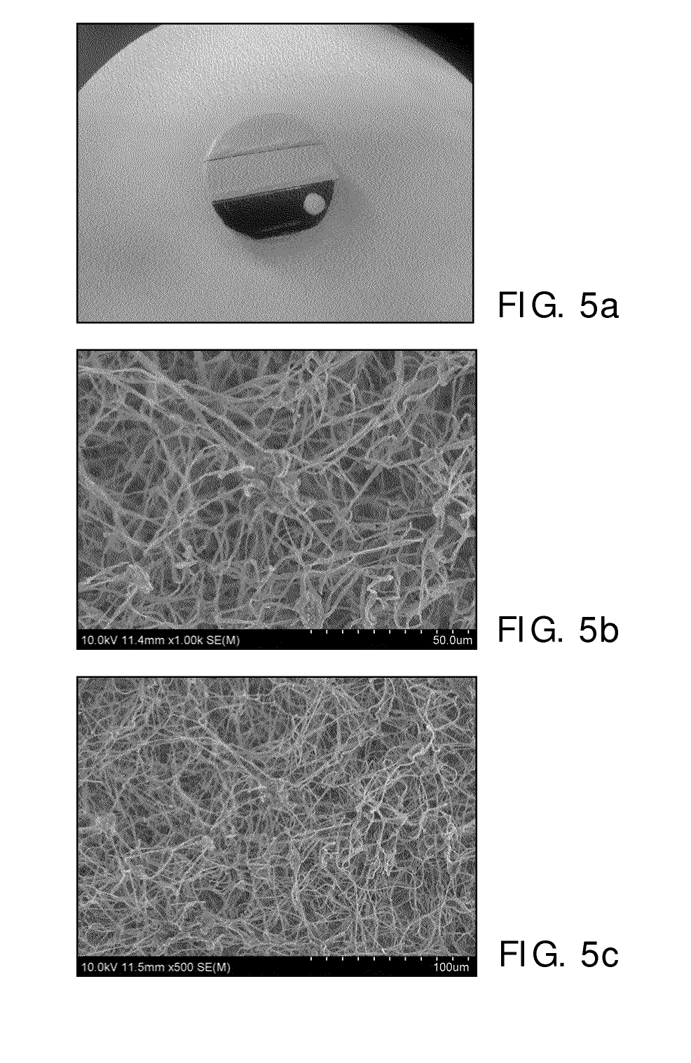

The surface morphology of the superhydrophobic coating as obtained above has been investigated through SEM. The sample preparation is shown in FIG. 5a, and the results are shown in FIGS. 5b and 5c in different magnifications.

It can be seen from FIGS. 5b and 5c that a structure of intertwined and entangled filaments was formed on the surface of the superhydrophobic coating. Without wishing to be bound by theory, it is believed that the intertwined and entangled filaments make it possible to entrap microscopic pockets of air therein and thereby support the water droplet in the form of a ball of water. The filaments were formed by coating and replicating the hyphae present in the streptomyces albus.

It should be noted that the above-mentioned embodiments illustrate rather than limit the invention, and that those skilled in the art will be able to design many alternative embodiments without departing from the scope of the appended claims. Although claims have been formulated in this application to particular combinations of features, it should be understood that the scope of the present disclosure also includes any novel features or any novel combinations of features disclosed herein either explicitly or implicitly or any generalization thereof, whether or not it relates to the same invention as presently claimed in any claim. The applicants hereby give notice that new claims may be formulated to such features and/or combinations of features during the prosecution of the present application or of any further application derived therefrom.

1. A method of treating a surface of a substrate, the method comprising:
a) growing a microbe on the surface of the substrate to be treated to form a layer of microbial structures, wherein the microbe is selected from fungi, bacteria, algae, lichens and any combination thereof; and
b) coating the microbial structures to form a first coating thereon, wherein the first coating has a thickness of no more than 1 μm.

2. A method according to claim 1, wherein the microbe is selected from fungi or bacteria comprising hyphae with a diameter of between 0.3 μm and 100 μm.

3. A method according to claim 2, wherein the fungi or bacteria are selected from Molds, Actinomycetes and any combination thereof.

4. A method according to claim 3, wherein the fungi or bacteria are selected from Rhizopus, Mucor, Neurospora, Aspergillus, Penicillium, Streptomyces, Nocardia, Frankia, Actinoplanes, thermomonospora and any combination thereof.

5. A method according to claim 4, wherein the Streptomyces is selected from Streptomyces albus, Streptomyces griseus, Streptomyces venezuelae, Streptomyces aureofaciens and any combination thereof.

6. A method according to claim 1, wherein the first coating is formed on the microbial structures by one or more methods selected from vapor deposition, self-propagation high-temperature synthesis, thermochemical synthesis, thermal spraying, electrochemical synthesis, sol-gel process, and in situ formation.

7. A method according to claim 1, wherein the first coating is an organic coating or an inorganic coating, and forms a shell around the microbial structures.

8. A method according to claim 1, wherein the first coating is a coating consisting of an inorganic oxide selected from silicon oxides, magnesium oxides, aluminum oxides, zinc oxides, iron oxides, copper oxides, silver oxides, titanium oxides, and any combination thereof.

9. A method according to claim 1, the method further comprising:
c) forming a second coating on the first coating.

10. A method according to claim 9, wherein the second coating is a coating consisting of hydrophobic materials, oleophobic materials, or amphiphobic materials, and the second coating has a surface energy of no more than 70 mJ/m2.

11. A method according to claim 10, wherein the second coating is selected from an organofluorine coating, an organosilicone coating, a fluorosilicone coating, and any combination thereof.

12. A method according to claim 9, wherein the microbe comprises Streptomyces albus, the first coating comprises silicon dioxide, and the second coating comprises a fluorinated silane.

13. A method according to claim 1, the method further comprising a step of deactivating or removing the microbe after the first coating has been formed.

14. An article comprising a substrate, a layer of microbial structures formed on at least a portion of a surface of the substrate, and a first coating formed on the microbial structures, wherein the microbial structures comprise a structure originating from fungi, algae, lichens or any combination thereof; and wherein the first coating has a thickness of no more than 1 μm.

15. An article according to claim 14, wherein the article is made by using a method for treating at least a portion of the surface of the substrate.
